(19) United States
(12) Patent Application Publication
(10) Pub. No.: US 2011/0251621 A1
(43) Pub. Date: Oct. 13, 2011

(54) DRILL SHAFT WITH OFFSET GUIDE

(75) Inventors: Robert Sluss, Naples, FL (US); John Christoforetti, Pittsburg, PA (US)

(73) Assignee: Arthrex, Inc., Naples, FL (US)

(21) Appl. No.: 13/085,600

(22) Filed: Apr. 13, 2011

Related U.S. Application Data
(60) Provisional application No. 61/323,598, filed on Apr. 13, 2010.

(51) Int. Cl. A61B 17/17

(52) U.S. Cl. 606/96

(57) ABSTRACT

A drill guide formed by a cannulated body with a distal opening and at least one drill guide arm extending from the outer wall forming the cannulated body at an acute angle is disclosed. The inner channel formed by the cannulated body may be concentric with the cannulated body. The drill guide arm may extend at an angle from the outer wall and may extend from the outer wall at a point generally opposite to a distalmost point on the distal surface. As such, the drill guide arm may be offset from the inner channel forming the cannulated body, thereby enabling the inner channel to be aligned and concentric with the cannulated body forming the inner channel.

### CROSS-REFERENCE TO RELATED APPLICATION

This patent application claims the benefit of U.S. Provisional Patent Application No. 61/323,598, filed Apr. 13, 2010, which is incorporated by reference in its entirety.

### FIELD OF THE INVENTION

The invention relates to drill guides useful in arthroscopy, and more particularly, to drill guides for implanting a suture anchor into a tissue in hip arthroscopy.

### BACKGROUND

During arthroscopic hip surgery, it is necessary to attach a suture into a rim of an acetabulum so that a surgeon may tie the suture to a labrum to secure it to the bone. Securing the suture can be problematic and damage can occur to the articular cartilage adjacent to the acetabluar rim. While it is desirable to attach to the suture to the acetabular rim, care must be taken to drill into the acetabular rim without perforating the acetabular rim, thereby protecting the articular cartilage and joint from accidental perforation with the drill. Thus, a drill guide is needed to guide a drill into contact with an acetabular rim but prevent accidental perforation of the acetabular rim and damage to the articular cartilage and joint.

### SUMMARY OF THE INVENTION

This invention is directed to a drill guide that is formed from a cannulated body with one or more drill guide arms extending distally from a distal end of the cannulated body. The drill guide arm enables one or more holes to be safely drilled in an acetabular rim without allowing the holes to be drilled completely through the acetabular rim. The drill guide arm may function as a depth gauge to help a user to accurately drill a hole in the acetabular rim. A proximalmost point of the distal end of the cannulated body may be offset toward the drill guide arm from a longitudinal axis of the cannulated body, thereby enabling the drill guide to create a socket on a rim of the acetabulum without drilling through the rim.

The drill guide may be formed from a cannulated body with a distal opening. A distal surface of the cannulated body at the distal opening may be positioned at an acute angle relative to a longitudinal axis of the cannulated body. The drill guide may include at least one first side vent orifice in an outer wall forming the cannulated body and proximate to the distal opening of the cannulated body and at least one second side vent orifice in the outer wall forming the cannulated body and proximate to the distal opening of the cannulated body. The first and second side vent orifices of the drill guide may be aligned.

The drill guide may include at least one drill guide arm offset from the longitudinal axis and extending from the outer wall forming the cannulated body at an acute angle relative to the longitudinal axis and extending from the outer wall at a point generally opposite to a distalmost point on the distal surface of the cannulated body, wherein a distalmost point on the at least one drill guide arm extends further distally than the distalmost point on the distal surface. The distalmost point on the distal surface may be inline with a linear axis extending along the outer wall of the cannulated body. The drill guide may include a transition section extending from a linear distal surface of the distal opening to the at least one drill guide arm. The transition section may be curved forming a mouth to cradle an acetabular rim. The drill guide arm may be a depth gauge such that a distance between the distal surface of the cannulated body at the distal opening and the distalmost point on the drill guide arm functions as a depth gauge.

The drill guide may include a third side vent orifice in the outer wall forming the cannulated body. The third side vent orifice may be positioned generally orthogonal to the first and second side vent orifice in the outer wall. The drill guide may also include a recess in the outer wall and wherein the at least one third side vent orifice is positioned in the recess.

The drill guide arm may extend generally linearly from the outer wall of the cannulated body. The drill guide arm may have rounded outer edges. The drill guide arm may be formed from a generally flat material. The drill guide arm may have a width equal to a width of the cannulated body.

An advantage of the invention is that the drill guide arm extends from the cannulated body such that the drill guide arm functions as a depth gauge.

Another advantage of the invention is that a proximalmost point of the distal end of the cannulated body may be offset toward the drill guide arm from a longitudinal axis of the cannulated body, thereby enabling the drill guide to create a socket on a rim of the acetabulum without drilling through the rim.

Yet another advantage of the invention is that the drill guide arm includes a curved mouth configured to cradle an acetabular rim such that the drill guide arm is positioned on the cartilage side of the acetabular rim to protect the acetabular rim.

Another advantage of the invention is that the first and second side vents enable bone chips to be removed from inner aspects of the cannulated body;

Still another advantage of the drill guide is that the side vents enable one to visually confirm the position of the drill guide on an acetabular rim.

Another advantage of the drill guide is that the drill guide arm is angled relative to a longitudinal axis of the cannulated tube such that that the drill guide does not position the drill to drill a hole through the acetabular rim but instead allows a user to visualize the depth and direction of where the drill will go, thereby enabling the user to make adjustments before drilling to prevent unintended damage.

These and other embodiments are described in more detail below.

### BRIEF DESCRIPTION OF THE FIGURES

FIG. 1 is a perspective view of portals and an associated acetabular rim of a human hip.

FIG. 2 is a perspective view of a distal end of a drill guide.

FIG. 3 is a side view of the distal end of the drill guide shown in FIG. 2.

FIG. 4 is partial cross-sectional view of a human hip with the drill guide engaged with an acetabular rim.

FIG. 5 is a photograph of a drill guide with a straight cannulated body and a drill guide with a curved cannulated body.

FIG. 6 is a detailed photograph of the drill guide with a straight cannulated body of FIG. 5 and the drill guide with a curved cannulated body of FIG. 5.

### DETAILED DESCRIPTION OF THE FIGURES

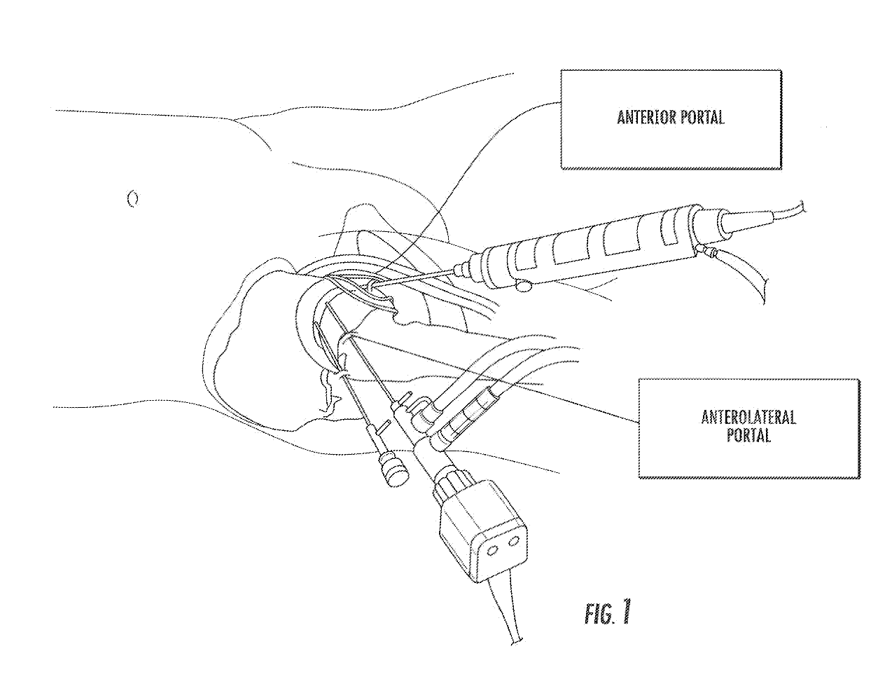

As shown in FIGS. 1-6, this invention is directed to a drill guide 10 that is formed from a cannulated body 12 with one or more drill guide arms 14 extending distally from a distal end of the cannulated body 12. The drill guide arm 14 enables one or more holes to be safely drilled in an acetabular rim 48 without allowing the holes to be drilled completely through the acetabular rim 48. A proximalmost point 21 of the distal end 26 of the cannulated body 12 may be offset toward the drill guide arm 14 from a longitudinal axis 46 of the cannulated body 12, thereby enabling the drill guide 10 to create a socket on a rim of the acetabulum without drilling through the rim.

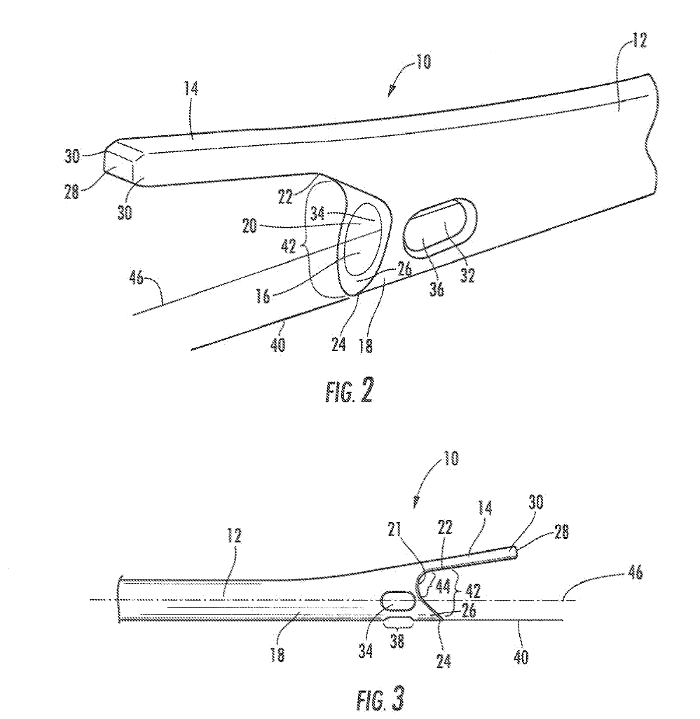

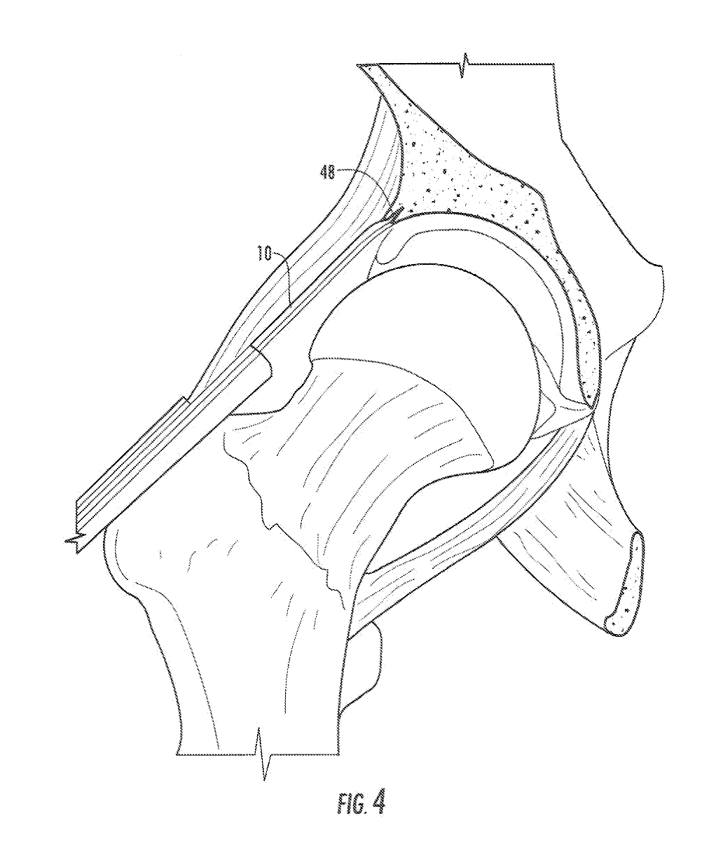

As shown in FIGS. 2 and 3, the drill guide 10 may be formed from a cannulated body 12 with a distal opening 16 and one or more drill guide arms 14 extending from an outer wall 18 forming the cannulated body 12 at an acute angle.

The drill guide arms 14 may extend at an acute angle from the outer wall 18 relative to the longitudinal axis 46 and may extend from the outer wall 18 at a point 22 generally opposite to a distalmost point 24 on the distal surface 26. As such, the drill guide 10 may be offset from an inner channel 20 forming the cannulated body 12, thereby enabling the inner channel 20 to be aligned and concentric with the cannulated body 12 forming the inner channel 20. As shown in FIG. 3, a proximalmost point 21 of the distal end 26 of the cannulated body 12 may be offset toward the drill guide arm 14 from a longitudinal axis 46 of the cannulated body 12, thereby enabling the drill guide 10 to create a socket on a rim of the acetabulum without drilling through the rim.

The drill guide arm 14 may extend generally linearly from the outer wall 18 of the cannulated body 12. The drill guide arm 14 may have rounded outer edges 30 to reduce the likelihood of injury to a patient. The drill guide arm 14 may be formed from a generally flat structure. The distalmost point 28 on the drill guide arm 14 may extend further distally than the distalmost point 24 on the distal surface 26. The distalmost point 24 on the distal surface 26 may be inline with a linear axis 40 extending along the outer wall 18 of the cannulated body 12.

The drill guide 10 may include one or more first side vent orifices 32 in an outer wall 18 forming the cannulated body 12 and proximate to the distal opening 16 of the cannulated body 12. The drill guide 10 may also include one or more second side vent orifices 34 in the outer wall 18 forming the cannulated body 12 and proximate to the distal opening 16 of the cannulated body 12. The first and second side vent orifices 32, 34 may be aligned and may be generally opposite to each other. In at least one embodiment, the first and second side vent orifices 32, 34 may have any appropriate shape. In at least one embodiment, the first and second orifices 32 may have generally elongated oval shapes. The drill guide 10 may also include a third side vent orifice 36 in the outer wall 18. The third side vent orifice 36 may be positioned generally orthogonal to both the first and second side vent orifices 32, 34 in the outer wall 18. The third side vent orifice 36 may have any appropriate shape. In at least one embodiment, the third side vent orifice 36 may have a generally elongated oval shape. The third side vent orifice 36 may be positioned in a recess 38 in the outer wall 18.

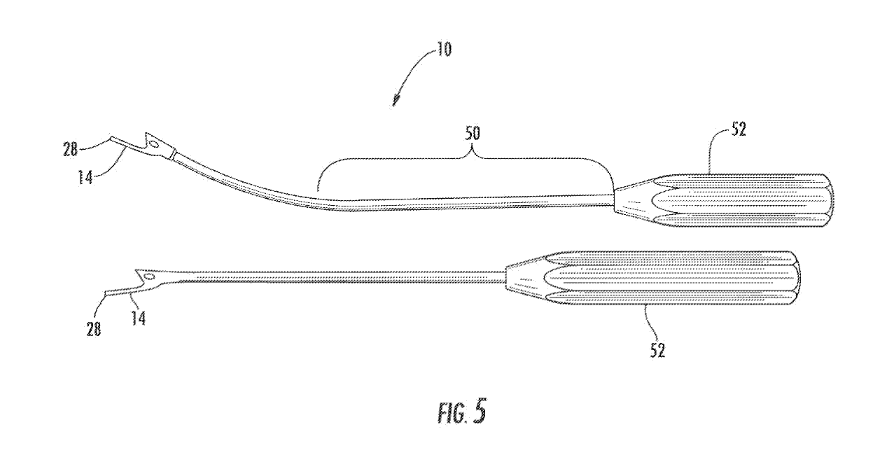

The distal surface 26 of the cannulated body 12 may be configured together with the drill arm 14 such that a recessed cavity 42 is formed. In particular, the drill arm 14 and the distal surface 26 of the cannulated body 12 may extend oppositely from each other to form the recessed cavity 42. A transition section 44 may extend from a linear distal surface 26 of the distal opening 16 to the drill guide arm 14. The transition section 44 may be curved forming a curved mouth configured to cradle an acetabular rim 48, as shown in FIG. 4. The drill guide arm 14 may have a width equal to a width of the cannulated body 12. As shown in FIGS. 2 and 3, the relationship of the drill arm 14 to the linear distal surface 26 may be such that the acetabular rim 48 is easily received between the drill arm 14 and the linear distal surface 26. In one embodiment, the drill guide arm 14 may extend from the cannulated body 12 between about five and about 45 degrees relative to a longitudinal axis 46, and the linear distal surface 26 may be positioned between about 20 degrees and about 60 degrees relative to the longitudinal axis 46. The position of the drill arm 14 relative to the longitudinal axis 46 prevents drilling through the acetabular rim 48. The drill guide arm 14 may function as a depth gauge as well. In particular, a distance between the distal surface 26 of the cannulated body 12 at the distal opening 16 and the distalmost point 24 on the drill guide arm 14 functions as a depth gauge. The drill guide arm 14 during use may be positioned in a patient such that the drill guide arm 14 protrudes past the acetabular rim and into the joint surface, thereby allowing a user to visualize the direction the drill will travel and protect the articular cartilage and joint from accidental perforation with the drill.

The drill arm 14 may function as a visual aid and may be used to position the cannulated body 12 correctly relative to the acetabular rim 48 such that a hole may be drilled correctly in the acetabular rim 48. As shown in FIG. 1, the drill guide 10 may be configured to access an acetabular rim 48 through an anterior portal, such as between a 12 o'clock and a 3 o'clock position, and an anterolateral portal, such as between a 10 o'clock and a 12 o'clock position.

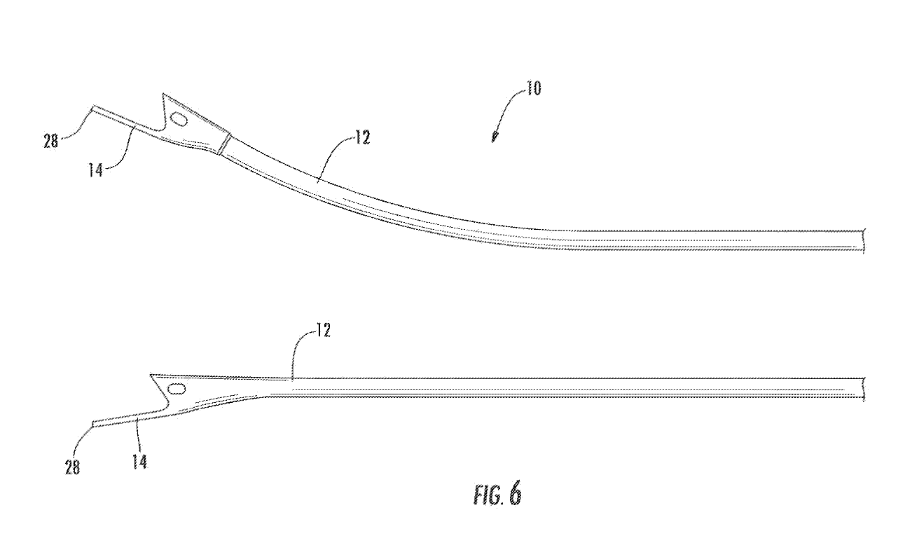

As shown in FIGS. 5 and 6, the cannulated body 12 may have different shapes. For instance, but not by way of limitation, the cannulated body 12 may be curved or straight. The curved cannulated body 12 may accommodate a flexible drill and flexible anchor insertion tool. The curved cannulated body 12 makes positioning the drill guide 10 easier because it does not require the surgeon to lever or push down the drill guide 10 against the patient's skin to direct the tip 28 up to the appropriate placement site on the acetabular rim through a standard anterior portal. Furthermore, the curved cannulated body 12 makes positioning the drill guide 10 easier because it does not require that a surgeon over torque the drill guide through a standard anterior portal. The curved cannulated body 12 may have an appropriate amount of curvature. In particular, the curved cannulated body 12 may be curved such that the tip 28 on the drill arm 14 is positioned between about 10 degrees and about 60 degrees from a linear portion 50 of the drill guide 10 extending from a handle 52. The curved cannulated body 12 may be curved such that an inner side of the curved portion of the cannulated body 12 is opposite to the side from which the drill arm 14 extends.

The foregoing is provided for purposes of illustrating, explaining, and describing embodiments of this invention. Modifications and adaptations to these embodiments will be apparent to those skilled in the art and may be made without departing from the scope or spirit of this invention.

We claim:

1. A drill guide, comprising:
a cannulated body with a distal opening, wherein a distal surface of the cannulated body at the distal opening is positioned at an acute angle relative to a longitudinal axis of the cannulated body;
at least one first side vent orifice in an outer wall forming the cannulated body and proximate to the distal opening of the cannulated body;
at least one second side vent orifice in the outer wall forming the cannulated body and proximate to the distal opening of the cannulated body; and
at least one drill guide arm offset from the longitudinal axis and extending from the outer wall forming the cannulated body at an acute angle relative to the longitudinal axis and extending from the outer wall at a point generally opposite to a distalmost point on the distal surface of the cannulated body, wherein a distalmost point on the at least one drill guide arm extends further distally than the distalmost point on the distal surface; and
wherein a proximalmost point of the distal end of the cannulated body is offset toward the at least one drill guide arm from a longitudinal axis of the cannulated body, thereby enabling the drill guide to create a socket on a rim of the acetabulum without drilling through the rim.

2. The drill guide of claim 1, wherein the at least one first and second side vent orifices are aligned.

3. The drill guide of claim 2, further comprising at least one third side vent orifice in the outer wall forming the cannulated body, wherein the at least one third side vent orifice is positioned generally orthogonal to the at least one first and second side vent orifice in the outer wall.

4. The drill guide of claim 3, further comprising a recess in the outer wall and wherein the at least one third side vent orifice is positioned in the recess.

5. The drill guide of claim 1, wherein the distalmost point on the distal surface is inline with a linear axis extending along the outer wall of the cannulated body.

6. The drill guide of claim 1, further comprising a transition section extending from a linear distal surface of the distal opening to the at least one drill guide arm.

7. The drill guide of claim 6, wherein the transition section is curved forming a mouth to cradle an acetabular rim.

8. The drill guide of claim 1, wherein the at least one drill guide arm extends generally linearly from the outer wall of the cannulated body.

9. The drill guide of claim 1, wherein the at least one drill guide arm has rounded outer edges.

10. The drill guide of claim 1, wherein a distance between the distal surface of the cannulated body at the distal opening and the distalmost point on the at least one drill guide arm functions as a depth gauge.

11. The drill guide of claim 1, wherein the at least one drill guide arm is formed from a generally flat material.

12. The drill guide of claim 1, wherein the at least one drill guide arm has a width equal to a width of the cannulated body.

13. A drill guide, comprising:
a cannulated body with a distal opening, wherein a distal surface of the cannulated body at the distal opening is positioned at an acute angle relative to a longitudinal axis of the cannulated body;
at least one first side vent orifice in an outer wall forming the cannulated body and proximate to the distal opening of the cannulated body;
at least one second side vent orifice in the outer wall forming the cannulated body and proximate to the distal opening of the cannulated body;
at least one drill guide arm offset from the longitudinal axis and extending from the outer wall forming the cannulated body at an acute angle relative to the longitudinal axis and extending from the outer wall at a point generally opposite to a distalmost point on the distal surface of the cannulated body, wherein a distalmost point on the at least one drill guide arm extends further distally than the distalmost point on the distal surface;
wherein a proximalmost point of the distal end of the cannulated body is offset toward the at least one drill guide arm from a longitudinal axis of the cannulated body, thereby enabling the drill guide to create a socket on a rim of the acetabulum without drilling through the rim;
wherein the at least one drill guide arm extends generally linearly from the outer wall of the cannulated body;
a transition section extending from a linear distal surface of the distal opening to the at least one drill guide arm; and
wherein the transition section is curved forming a mouth to cradle an acetabular rim.

14. The drill guide of claim 13, wherein the at least one first and second side vent orifices are aligned and the at least one drill guide arm has rounded outer edges.

15. The drill guide of claim 14, further comprising at least one third side vent orifice in the outer wall forming the cannulated body, wherein the at least one third side vent orifice is positioned generally orthogonal to the at least one first and second side vent orifice in the outer wall.

16. The drill guide of claim 15, further comprising a recess in the outer wall and wherein the at least one third side vent orifice is positioned in the recess.

17. The drill guide of claim 13, wherein the distalmost point on the distal surface is inline with a linear axis extending along the outer wall of the cannulated body.

18. The drill guide of claim 13, wherein a distance between the distal surface of the cannulated body at the distal opening and the distalmost point on the at least one drill guide arm functions as a depth gauge.

19. The drill guide of claim 13, wherein the at least one drill guide arm is formed from a generally flat material and wherein the at least one drill guide arm has a width equal to a width of the cannulated body.

20. A drill guide, comprising:
a cannulated body with a distal opening, wherein a distal surface of the cannulated body at the distal opening is positioned at an acute angle relative to a longitudinal axis of the cannulated body;
at least one first side vent orifice in an outer wall forming the cannulated body and proximate to the distal opening of the cannulated body;
at least one second side vent orifice in the outer wall forming the cannulated body and proximate to the distal opening of the cannulated body;
at least one third side vent orifice in the outer wall forming the cannulated body, wherein the at least one third side vent orifice is positioned generally orthogonal to the at least one first and second side vent orifice in the outer wall;
at least one drill guide arm offset from the longitudinal axis and extending from the outer wall forming the cannulated body at an acute angle relative to the longitudinal axis and extending from the outer wall at a point generally opposite to a distalmost point on the distal surface of the cannulated body, wherein a distalmost point on the at least one drill guide arm extends further distally than the distalmost point on the distal surface;
wherein a proximalmost point of the distal end of the cannulated body is offset toward the at least one drill guide arm from a longitudinal axis of the cannulated body, thereby enabling the drill guide to create a socket on a rim of the acetabulum without drilling through the rim;
wherein the at least one drill guide arm extends generally linearly from the outer wall of the cannulated body;
a transition section extending from a linear distal surface of the distal opening to the at least one drill guide arm;
wherein the transition section is curved forming a mouth to cradle an acetabular rim;
wherein the at least one drill guide arm is formed from a generally flat material and wherein the at least one drill guide arm has a width equal to a width of the cannulated body; and
wherein a distance between the distal surface of the cannulated body at the distal opening and the distalmost point on the at least one drill guide arm functions as a depth gauge.
